(19) United States
(12) Patent Application Publication
(10) Pub. No.: US 2018/0000750 A1
(43) Pub. Date: Jan. 4, 2018

(54) ANTIPLATELET AGENT AND USES THEREOF

(71) Applicants: Wei-Kung TSENG, Taichung City (TW); Chia-Chun HSU, Kaoshiung City (TW); Wan-Lin WU, Taipei (TW)

(72) Inventors: Wei-Kung TSENG, Taichung City (TW); Chia-Chun HSU, Kaoshiung City (TW); Wan-Lin WU, Taipei (TW)

(21) Appl. No.: 15/541,714

(22) PCT Filed: Jan. 8, 2016
(86) PCT No.: PCT/CN2016/070467

§ 371 Date: Jul. 6, 2017

Related U.S. Application Data
(60) Provisional application No. 62/100,929, filed on Jan. 8, 2015.

(51) Int. Cl. A61K 31/122; A61K 31/519; A61K 31/366; A61K 31/4365; A61K 31/616; A61K 31/5375

(52) U.S. Cl. CPC A61K 31/122; A61K 31/616; A61K 31/5375; A61K 31/366; A61K 31/4365; A61K 31/519

(57) ABSTRACT

Disclosed herein are methods for suppressing or inhibiting platelet aggregation in a subject in need thereof. The method includes administering to the subject in need thereof an effective amount of Physalin to alleviate or ameliorate symptoms associated with diseases, disorders, and/or conditions resulted from platelet aggregation. According to preferred embodiments, Physalin is applied as a coating on an implantable device, such as a stent or a catheter.

### CROSS-REFERENCE TO RELATED APPLICATION

This application relates to and claims the benefit of U.S. Provisional Application No. 62/100,929, filed Jan. 8, 2015, the contents of which is incorporated herein by reference in their entirety.

### BACKGROUND OF THE INVENTION

### 1. Field of the Invention

The present disclosure in general relates to platelet aggregation inhibitors and methods of using the same. The methods are advantageously useful for decreasing or preventing platelet aggregation and platelets activation in a subject or a biological sample.

### 2. Description of Related Art

Platelets are involved in many physiologic and pathological processes such as atherothrombosis, stem cell trafficking, tumor metastasis, and arthritis. Platelet activation at sites of an intact inflamed endothelium contributes to vascular inflammation and vascular wall remodeling. Platelets interact with the vascular endothelium and link the processes of inflammation, thrombosis, and atherogenesis, which is mediated through the interactions between platelets and endothelial cells/leukocytes. Platelets can induce a variety of inflammatory responses in monocytes, neutrophils (PMN), endothelial cells, or endothelial progenitor cells (EPCs), resulting in key inflammatory processes, such as adhesion, chemotaxis, migration, thrombosis, or even monocytic cell differentiation to macrophages or foam cells. EPCs are pluripotent cells that differentiate into mature endothelial cells. Previous studies have demonstrated that healthy persons have a small number of circulating EPCs in the peripheral blood (Hill et al., 2003 N Engl J Med 348, 593-600). In recent years, investigations have focused on elucidating the cellular mechanisms of EPCs on vasculogenesis in order to find new methods to alleviate certain cardiovascular disease conditions (Roberts et al., 2005 J Cell Mol Med, 9, 583-591.). The level of circulating EPCs was regarded as a marker for the prognosis of acute coronary events (Werner and Nickenig, 2006, Arterioscler Thromb Vasc Biol 26, 257-266.), and served as a biologic index for vascular function and cardiovascular risk.

Platelet activation plays an important role in the process of inflammation and the initiation of atherosclerosis. Many cardiovascular diseases (CVDs), including the initiation of atherothrombosis, are linked to the abnormal and excessive activation of platelets, or platelet hyperactivity, which is considered an independent risk factor for CVDs. Acetylsalicylic acid (aspirin) was the first antiplatelet agent identified, which irreversibly inhibits the cyclooxygenase 1 (COX1) enzyme in the arachidonic acid pathway through acetylation of the COX1 active site. Long-term aspirin therapy reduces the risk of subsequent myocardial infarction, stroke or vascular death among intermediate to high-risk patients with atherothrombotic disease by about 20%-25% (Patrono et al., 2004 Chest 126, 234S-264S). However, bleeding risk is a substantial limitation of antiplatelet therapy. On the other hand, the thienopyridines (ticlopidine and clopidogrel) target platelet activation pathways critical for both protective hemostasis and pathologic thrombosis, which can be detected clinically as a prolonged bleeding time (Scarborough et al., 1999 Circulation 100, 437-444). Though recent novel antiplatelet agents, including clopidogrel and ticagrelor, provide potent antiplatelet effect on CVD therapy, bleeding remained an important clinical issue. Scientists are still working on the balance between bleeding and efficacy for a safe antiplatelet agent.

Activated platelets stimulate thrombus formation in response to atherosclerotic plaque rupture or endothelial erosion, thereby promoting atherothrombotic events. Activated platelets also interact with the endothelial cells and leukocytes to promote inflammation, which contribute to atherosclerosis. Antiplatelet drugs therefore are important in cardiovascular disease therapy. Clopidogrel, a thienopyridine, combined with aspirin, is the current “gold standard” for reducing cardiovascular events in acute coronary syndrome (ACS) patients. However, not all patients respond optimally to this standard therapy. When used either singly or in combination, resistance to the antiplatelet activity of both drugs occurs, possibly leading to treatment failure including additional atherothrombotic events. Besides, bleeding risk is always a major clinical concern when these antiplatelet therapy are applied. Thus, developing a more effective and safer new drug for antiplatelet aggregation is necessary.

In view of the above, there exists in the related art a need of an agent that suppresses or inhibits the aggregation and/or activation of platelets without the bleeding risk concern, thus may serve as a potential lead compound for the development of a medicament for treating diseases, disorders, and/or conditions resulted from platelet aggregation.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In general, the present disclosure relates to the unexpected discovery of the novel use of Physalin B in suppressing platelet aggregation and platelet activation. Thus, Physalin B may act as a potential lead compound for developing medicaments for treating diseases and/or conditions resulted from platelet aggregation.

Accordingly, the first aspect of the present disclosure aims at providing a use of Physalin B in manufacturing a medicament for the treatment of a disease resulting from platelet aggregation or blood coagulation.

According to embodiments of the present disclosure, the disease and/or condition resulting from platelet aggregation is a thrombotic disorder, which may be selected from the group consisting of, abrupt vessel closure following angioplasty or stent placement, atherothrombosis, acute thrombotic stroke, myocardial infarction, thrombosis resulted from periphery vascular surgery, unstable angina, and venous thrombosis.

According to preferred embodiment of the present disclosure, the thrombotic disorder is atherothrombosis.

According to further embodiments of the present disclosure, the medicament further comprises an anti-coagulant, which may be selected from the group consisting of, abciximab, apixaban, aspirin, clopidogrel, dipyridamole, edoxaban, eptifibatide, rivaroxaban, tirofiban, ticlopidine, warfarin, and vitamin K.

According to preferred embodiments, Physalin B is applied as a coating on the surface of an implantable device, which includes and is not limited to, a stent and a catheter. Optionally, Physalin B and the anti-coagulant are respectively applied as coatings on the surface of the implantable device.

The second aspect of the present disclosure aims at providing a method of treating a subject having or suspected of having a disease and/or a condition resulting from platelet aggregation. The method comprises administering to the subject an effective amount of Physalin B to alleviate or ameliorate the symptoms associated with the disease and/or condition resulting from platelet aggregation.

According to embodiments of the present disclosure, the Physalin B is administered to the subject in the amount of 0.001-100 mg/Kg. Preferably, the Physalin B is administered to the subject in the amount of 0.001-10 mg/Kg; more preferably, the Physalin B is administered to the subject in the amount of 0.01-10 mg/Kg.

According to embodiments of the present disclosure, the disease and/or condition resulting from platelet aggregation is a thrombotic disorder, which may be selected from the group consisting of, abrupt vessel closure following angioplasty or stent placement, atherothrombosis, acute thrombotic stroke, myocardial infarction, thrombosis resulted from periphery vascular surgery, unstable angina, and venous thrombosis.

According to preferred embodiment of the present disclosure, the thrombotic disorder is atherothrombosis.

According to embodiments of the present disclosure, the method further comprises administering to the subject an anti-coagulant, which may be selected from the group consisting of, abciximab, apixaban, aspirin, clopidogrel, dipyridamole, edoxaban, eptifibatide, rivaroxaban, tirofiban, ticlopidine, warfarin, and vitamin K.

According to preferred embodiments of the present disclosure, Physalin B is applied as a coating on the surface of an implantable device, which includes and is not limited to, a stent and a catheter. Optionally, Physalin B and the anti-coagulant are respectively applied as coatings on the surface of the implantable device.

Accordance to embodiments of the present disclosure, the subject is human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 illustrates the effects of Physalin B on platelet aggregation in accordance with one embodiment of the present disclosure, in which EPI: activated by collagen and epinephrine; ADP: activated by collagen and ADP; and * and # indicated P<0.05 by t-test compared to the control values. Data was obtained from 10 healthy blood samples;

FIG. 2 illustrates the effects of Physalin B on the tail bleeding time in accordance with one embodiment of the present disclosure, in which each symbols represents the bleeding time of one individual animal, data was presented as the mean±S.E.M. (n=10), *p<0.05 compared with the DMSO group.

FIG. 3 is a graph illustrating the effects of Physalin B on the occlusion time for irradiation induced platelet plug formation in accordance with one embodiment of the present disclosure, in which each symbols represents the occlusion time of one individual animal, data was presented as the mean±S.E.M. (n=6), ***p<0.001 compared with the DMSO group.

FIG. 4 illustrates the cytotoxic effects of Physalin B on platelets in accordance with one embodiment of the present disclosure;

FIG. 5A illustrates the effects of Physalin B on the viability of HUVECs in accordance with one embodiment of the present disclosure;

FIG. 5B illustrates the effects of Physalin B on the viability of EPCs in accordance with another embodiment of the present disclosure; and

FIG. 6 is a bar graph depicting the effects of Physalin B on the adhesion of THP-1 monocytes to TNF-α-activated HUVECs in accordance with one embodiment of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definitions

For convenience, certain terms employed in the context of the present disclosure are collected here. Unless defined otherwise, all technical and scientific terms used herein have the same meaning as commonly understood by one of the ordinary skill in the art to which this invention belongs.

The term “treatment” as used herein are intended to mean obtaining a desired pharmacological and/or physiologic effect, e.g., delaying or inhibiting platelet aggregation and/or platelet activation. The effect may be prophylactic in terms of completely or partially preventing a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., a cancer or heart failure) from occurring in an individual who may be pre-disposed to the disease but has not yet been diagnosed as having it; (2) inhibiting a disease (e.g., by arresting its development); or (3) relieving a disease (e.g., reducing symptoms associated with the disease).

The term “administered”, “administering” or “administration” are used interchangeably herein to refer a mode of delivery, including, without limitation, intraveneously, intramuscularly, intraperitoneally, intraarterially, intracranially, or subcutaneously administering an agent (e.g., a compound or a composition) of the present invention. In some embodiments, the compound of the present disclosure (i.e., Physalin B) are formulated into powders for mixed with suitable carrier (e.g., buffer solution) before use, such as intraveneous injection. In other embodiments, the compound of the present disclosure (i.e., Physalin B) is directly applied or coated onto an angioplasty stent (e.g., a coronary stent or a vascular stent) or a stent graft for use in a vascular surgical procedure.

The term “an effective amount” as used herein refers to an amount effective, at dosages, and for periods of time necessary, to achieve the desired result with respect to the treatment of a disease resulted from platelet aggregation. For example, in the treatment of a thrombotic disorder, an agent (i.e., the present compound) which decrease, prevents, delays or suppresses or arrests any symptoms of the thrombotic disorder would be effective. An effective amount of an agent is not required to cure a disease or condition but will provide a treatment for a disease or condition such that the onset of the disease or condition is delayed, hindered or prevented, or the disease or condition symptoms are ameliorated. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the like. Effective amount may be expressed, for example, as the total mass of the active agent (e.g., in grams, milligrams or micrograms) or a ratio of mass of the active agent to body mass, e.g., as milligrams per kilogram (mg/kg). The effective amount may be divided into one, two or more doses in a suitable form to be administered at one, two or more times throughout a designated time period.

The term “subject” or “patient” is used interchangeably herein and is intended to mean a mammal including the human species that is treatable by the compound of the present invention. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, rat, mouse, guinea pig, monkey, pig, goat, cow, horse, dog, cat, bird and fowl. In a preferred embodiment, the subject is a human.

The term “pharmaceutically acceptable” refers to molecules and compositions that do not produce an adverse or undesirable reaction (e.g., toxicity, or allergic reaction) when administered to a subject, such as a human.

The term “excipient” as used herein means any inert substance (such as a powder or liquid) that forms a vehicle/carrier for the active agent. The excipient is generally safe, non-toxic, and in a broad sense, may also include any known substance in the pharmaceutical industry useful for preparing pharmaceutical compositions such as, fillers, diluents, agglutinants, binders, lubricating agents, glidants, stabilizer, colorants, wetting agents, disintegrants, and etc.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a”, “and”, and “the” are used herein to include plural referents unless the context clearly dictates otherwise.

### 2. Detail Description of Preferred Embodiments

The present disclosure is based, at least in part, on the unexpected discovery that Physalin B, a member of Physalins isolated from Physalis plants, may suppress or inhibit platelet aggregation. Accordingly, Physalin B may serve as a potential lead compound for the development of a medicament for treating diseases, disorders and/or conditions resulting from platelet aggregation.

The practices of this invention are hereinafter described in detail with respect to use of Physalin B, a composition comprising Physalin B, the preparation of a medicament for preventing or treating thrombosis, or disease caused thereby, in a subject or patient who is to undertake a surgical procedure. Results of the present studies, as described herein below, show that Physalin B possess minimum or no cytotoxicity toward platelets or epithelial cells, and impedes the adherence of platelets and/or monocytes onto vascular endothelia cells, thereby suppresses the aggregation or activation of platelets in vivo.

The first aspect of the present application is therefore directed to a method of treating a subject having or suffering from a disease, disorder and/or condition resulted from platelet aggregation. The method comprises the step of, administering to the subject in need thereof, an effective amount of physalin B, so as to alleviate or ameliorate the symptoms associated with the disease, disorder and/or condition resulted from platelet aggregation.

In some embodiments, the Physalin B may inhibit an epinephrine signaling pathway, thereby suppressing epinephrine-induced platelet aggregation. Epinephrine may activate the aggregation of platelets, particularly in subjects suffering from acute vascular disease, which includes but is not limited to, atherothrombosis, deep vein thrombosis, myocardial infarction, pulmonary embolism, peripheral arterial occlusion, stroke, unstable angina and other blood system thromboses.

In other embodiments, the Physalin B may prevent or inhibit undesired platelet aggregation in certain medical procedures, such as preventing platelets from aggregating following vascular surgery (e.g., angioplasty or stent placement).

According to some embodiments of the present disclosure, Physalin B may be administered to the subject intravenously, subcutaneously, or orally in the amount of 0.001-100 mg/Kg, preferably in the amount of 0.001-10 mg/Kg; more preferably in the amount of 0.01-10 mg/Kg; and most preferably in the amount of 2-8 mg/Kg.

According to other embodiments, the Physalin B is coated on the surface of an implantable device (e.g., a stent or a tube), which is then inserted into blood vessels, urinary tracts or other difficult to access places for the purpose of preventing restenosis, providing vessel or lumen wall support or reinforcement. In this regard, the Physalin B is preferably in the form of a solution or a suspension with Physalin B homogeneously dispersed therein. The coating is preferably applied as a plurality of relatively thin layers sequentially applied in relatively rapid sequence and is preferably applied with the stent in a radially expanded state. The coating may be applied by dipping or spraying using evaporative solvent materials of relatively high vapor pressure to produce the desired viscosity and quickly establish coating layer thicknesses. The coating process enables the Physalin B to adherently conform to and cover the entire surface of the open structure of the stent or the catheter.

According to optional embodiments, Physalin B may be used in conjugation with another anti-coagulant to treat diseases, disorders, and/or conditions resulted from the activation or aggregation of platelets. Anti-coagulant or platelet inhibitors suitable for use with Physalin B are, for example, glycoprotein IIb/IIIa antagonists, heparins, tissue plasminogen activators, Factor Xa inhibitors, thrombin inhibitors, phosphodiesteras inhibitors, cyclooxygenase inhibitors, and etc. Suitable examples of anti-coagulant that may be used in the present method include, and are not limited to, abciximab, apixaban, aspirin, clopidogrel, dipyridamole, edoxaban, eptifibatide, rivaroxaban, tirofiban, ticlopidine, warfarin, and vitamin K. In one example, clopidogrel is administered concurrently with Physalin B.

The second aspect of the present application is directed to a medicament or a pharmaceutical composition for treating a disease, disorder and/or condition resulted from platelet aggregation. The pharmaceutical composition comprises an effective amount of Physalin B, and a pharmaceutically acceptable excipient.

Generally, physalin B is present in the pharmaceutical composition at a level of about 0.01% to 99.9% by weight, based on the total weight of the pharmaceutical composition. In some embodiments, physalin B is present at a level of at least 0.1% by weight, based on the total weight of the pharmaceutical composition. In certain embodiments, physalin B is present at a level of at least 5% by weight, based on the total weight of the pharmaceutical composition. In still other embodiments, physalin B is present at a level of at least 10% by weight, based on the total weight of the pharmaceutical composition. In still yet other embodiments, physalin B is present at a level of at least 25% by weight, based on the total weight of the pharmaceutical composition.

In some embodiments, the pharmaceutical composition of this invention further includes an agent (e.g., anti-coagulant) known to alleviate or ameliorate the symptoms of the disease, disorder, and/or condition resulting from platelet aggregation. Examples of such agent include, and are not limited to, glycoprotein IIb/IIIa antagonists, heparins, tissue plasminogen activators, Factor Xa inhibitors, thrombin inhibitors, phosphodiesteras inhibitors, cyclooxygenase inhibitors, and etc. Suitable examples of anti-coagulant that may be used in the present method include, and are not limited to, abciximab, apixaban, aspirin, clopidogrel, dipyridamole, edoxaban, eptifibatide, rivaroxaban, tirofiban, ticlopidine, warfarin, and vitamin K. In one example, clopidogrel is administered concurrently with Physalin B.

Pharmaceutically acceptable excipients are those that are compatible with other ingredients in the formulation and biologically acceptable.

The pharmaceutical composition may comprise different types of excipients depending on the intended routes of administration. The present composition may be administered intraveneously, intradermally, intraarterially, intraperitoneally, intralesionally, intracranially, intranasally, intrapleurally, intratracheally, intrarectally, topically, intramuscularly, subcutaneoustly, intravesicularlly, intrapericardially, intraocularally, orally, topically, locally, injection, inhalation, infusion, localized perfusion, in any suitable forms such as powders, creams, liquids, aerosols and etc.

The actual dosage of the medicament or the pharmaceutical composition may be determined by the attending physician based on the physical and physiological factors of the subject, these factors include, but are not limited to, age, gender, body weight, the disease to be treated, severity of the condition, previous history, the presence of other medications, the route of administration and etc. According to non-limiting examples of the present disclosure, each dosage will give rise to 1-10 mg Physalin B/Kg body weight per administration.

The pharmaceutical compositions containing Physalin B may be in a form suitable for oral use, for example, as tablets, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain Physalin B in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc.

The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffin, or olive oil.

For example, a solid oral composition such as a tablet or capsule may contain from 1 to 99% (w/w) Physalin B; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) Physalin B; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilizers, tonicity adjusting agents and preservatives.

The pharmaceutical compositions of the invention may be in the form of an oil-in-water emulsion. The oily phase may be a vegetable oil, for example olive oil or peanut oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a preservative, and flavouring and colouring agents. The pharmaceutical compositions may be in the form of a sterile injectable aqueous or oleagenous suspension. This suspension may be formulated according to the known art using suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringers solution and isotonic sodium chloride solution. Co-solvents such as ethanol, propylene glycol or polyethylene glycols may also be used. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Physalin B may also be administered in the form of suppositories for rectal administration of the drug. These compositions can be prepared by mixing the drug with a suitable non-irritating excipient which is solid at ambient temperatures but liquid at the rectal temperature and will therefore melt in the rectum to release the drug. Such materials are cocoa butter and polyethylene glycols.

For topical use, creams, ointments, gels, solutions or suspensions, etc., containing Physalin B are employed. (For purposes of this application, topical application shall include mouth washes and gargles.) Topical formulations may generally be comprised of a pharmaceutical carrier, co-solvent, emulsifier, penetration enhancer, preservative system, and emollient.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent.

### EXAMPLES

Materials and Methods

Culture of Endothelial Progenitor Cells (EPCs)

Two different types of EPCs were cultured from adult peripheral blood and defined as early EPCs and late EPCs according to their time-dependent appearance. Late EPCs, exhibited long lifespan and rapidly proliferated and considered as mature endothelial cells. Peripheral blood (20 mL) was obtained from donors with informed consent. The mononuclear cells were fractionated from other components of peripheral blood by centrifugation on Histopaque 1077 (Sigma, St. Louis, Mo.), with gradients according to the manufacturer's instructions. The isolated mononuclear cells were re-suspended with a EGM-2 BulletKit system (catalog number CC-3162; Clonetics™) consisting of an endothelial basal medium, 5% fetal bovine serum, hEGF, VEGF, hFGF-B, IGF-1, ascorbic acid, and heparin; 1×10⁷ mononuclear cells per well were seeded on 2% gelatin-coated 6-well plates (Sigma, St. Louis, Mo.) and incubated in a 5% CO₂ incubator at 37° C. Under daily observation, the first media change was performed approximately 5 days after plating. Thereafter, media were changed every 3 days. Each cluster or colony was followed up every day. For all assays, late EPCs were used at passages 3-5.

Culture of Human Umbilical Vein Endothelial Cells (HUVECs) and Monocyte Cell Line THP-1

HUVECs were purchased from American Type Culture Collection (ATCC) and were cultivated in endothelal cell nutrient medium consisting of 20% heat-inactivated fetal bovine serum (FBS), 80% Medium 199 (M199) buffered with 25 mM HEPES and supplemented with 2 mM L-Glutamine and 100 U/ml K-Penicillin and 100 μg/mL streptomycin, and kept at 37° C. in a 5% CO₂ atmosphere.

THP-1 were cultivated in Dulbecco's modified Eagle's medium (DMEM) supplemented with 10% heat-inactivated fetal bovine serum (FBS), 4 mM L-glutamine, adjusted to contain 1.5 g/L sodium bicarbonate, 4.5 g/L glucose, and 100 U/mL penicillin, and 100 μg/mL streptomycin, and kept at 37° C. in a 5% CO₂ atmosphere.

Preparation of Physalin B

The dried whole plants of P. angulate were obtained from Tainan District Agricultural Research & Extension Station, COA, Tainan County, Taiwan. The dried plant (3.5 Kg) was extracted with methanol (OH) at room temperature and concentrated under reduced pressure. The MeOH extract (297 g) was partitioned between ethyl acetate (EtOAc) and water to yield EtOAc and water extract. These extracts were then evaporated to give dark-green viscous residues. The EtOAc extract was separated by silica gel column chromatography using a gradient of n-hexane-EtOAc-MeOH to yield 19 fractions. Fractions 8 (203.4 mg) and 9 (154.8 mg) were combined and chromatographed on a silica gel column using n-hexane-EtOAc (2:1) as eluting solvent to produce further 3 fractions. Fraction (8+9)-2 was subject to purification by preparative TLC using n-hexane-EtOAc (1:1) as the eluting solvent, and the products was recrystallized from MeOH to yield Physalin B (25.2 mg). The chemical structure of Physalin B and its purity (>98%) was confirmed using mass spectrometry (MS), and nuclear magnetic resonance (NMR).

Lactate Dehydrogenase Assays

Cytotoxicity of physalin B on platelets were evaluated by measuring the release of lactate dehydrogenase (LDH). Platelets were suspended in Tyrodes's solution at a concentration of 3×10⁸ platelets/ml. For positive control, platelets were disrupted by sonication at 4° C. for 30 sec at 40 kHz with a Biosonic Sonicator. The supernatant was used for LDH determinations. LDH activity was measured as an increase in the absorbance of NADH at 340 nm using lactate as substrates. The tests were carried out with a Toshiba Medical automatic chemical analyzer, TBA-200FR (Toshiba Medical products). The platelets were pretreated with various concentrations of physalin B for 30 min at 37° C., and the supernatant was used to measure the LDH activity.

MTT Assay for Cell Viability

Cell viability was measured with blue formazan that was metabolized from 3-(4,5-dimethylthiazol-2-yl)-2, 5-diphenyl tetrazolium bromide (MTT, Amresco USA) in mitochondria, which is active only in live cells. HUVEC and late EPCs were seeded in a 96-well plate at a density of 1×10⁵ cells per well, cultured overnight and pretreated with various concentrations of physalin B. After incubation for 6 hr, 24 hr and 48 hr, the MTT (5 mg/ml) colorimetric viability test was used to determine the viability of cells. The absorbance of each well was measured at 540 nm with an ELISA reader and the percentage viability was calculated.

Assay for THP-1 Cell Adhesion to HUVEC

HUVECs were starved in serum-free medium for 1 hour before treatments. For adhesion assays, HUVECs monolayers in 96-well plates were treated for 2 hours with physalin B and/or TNF-α for 4 hours. After treatment, 1×10⁶ THP-1 cells labeled with 5 μM calcein-AM were seeded onto confluent HUVECs and co-cultured for 30 min at 37° C. in 5% CO₂ incubator. Non-adherent THP-1 cells were removed by washing with 1× phosphate buffered saline (PBS) twice. Cell images were collected by a fluorescence microscope (Zeiss) and quantified using a fluorescence microplate reader at an excitation wavelength of 490 nm and an emission wavelength of 525 nm (Bio-Tek Synergy HT).

Determination of Platelet Function and Anticoagulation Assay

Ten healthy, non-smoking volunteers (6 women, 4 men), ages 23-39 years old, participated in this study. The citrated whole blood samples were incubated at room temperature for 60 min. with physalin B (dissolved in 0.5% DMSO) or the vehicle alone. Platelet function measurement was performed with a PFA-100 analyzer system (Dade-Behring, Marburg, Germany). The PFA-100TM device measures the closure time (CT) required for platelets to plug an aperture simulating an injured vessel after platelet activation by relevant stimuli, namely collagen-epinephrine (EPI) or collagen-ADP (ADP). The maximum value for closure time is 300 seconds and values greater than 300 seconds are reported as non-closure. The closure time was determined with duplicate samples of 800 μL using cartridges containing collagen-epinephrine or collagen-ADP membranes.

To further identify which platelet surface receptors were involved in the anti-platelet effect, the VerifyNow system (Accumetrics, San Diego, Calif., USA) was used. It is a whole-blood assay based on light transmission measurements. The assay is a turbidimetric-based optical detection system that, like optical aggregometry, depends on the ability of activated platelets to bind fibrinogen. When platelets are activated, they form aggregates with fibrinogen-coated beads and the light transmission through the samples increases. Arachidonic acid, ADP and thrombin receptor activating peptide are the corresponding agonists used in VerifyNow system to specifically differentiate the platelet activation pathways. In the aspirin-specific assay, the degree of aggregation attenuated by aspirin, which blocks the arachidonic acid pathway, is quantified according to a corresponding decrease in light transmission and is reported as aspirin reaction units (ARU). ARU value less than 550 indicates adequate platelet inhibition by aspirin treatment. The residual activity of platelet after inhibition on P2Y12 receptor is represented as the P2Y12 reaction units (PRU). The normal PRU distribution range without anti-platelet therapy is 194-418. P2Y12 antagonist like clopidogrel and ticagrelor may be used as the positive control. The residual platelet activity under the treatment of antagonist on glycoprotein (GP) IIb/IIIa receptors has been reported as platelet aggregation units (PAU). The reference range was 125-330. Aggrastat may be used as the positive control medication for PAU value.

Further, Sysmex CA-1500 (Sysmex, Japan) was used to determine various coagulation parameters, including activated partial thromboplastin time (aPTT), prothrombin time (PT) and the fibrinogen level with 3.2% sodium citrate plasma treated with physalin B or heparin. Platelet-poor-plasma was first incubated with physalin B or heparin at 37° C. for 7 min. Extrinsic factor activity (II, V, VII, X), intrinsic factor activity (VIII, IX, XI, XII) and fibrinogen concentration were measured with one-stage prothrombin time based assay (PT, Siemens, PT Innovin), one-stage activated partial thromboplastin based assay (APTT, Siemens, Actin FSL) and Clauss Method (Siemens, Dade thrombin reagent), respectively.

Animals

ICR mice (7-8 weeks old, each weighted about 18 to 25 g) were used in this study. All mice were maintained in the animal facility under 12 hrs light/dark cycle, with ad libitum access to food and water.

### Example 1 Physalin B Inhibits Platelet Aggregation

In this example, effects of physalin B on platelet functions were respectively evaluated by use of 2 different assay systems, they were PFA-100 system, and VerifyNow P2Y12 system.

1.1 PFA-100 System

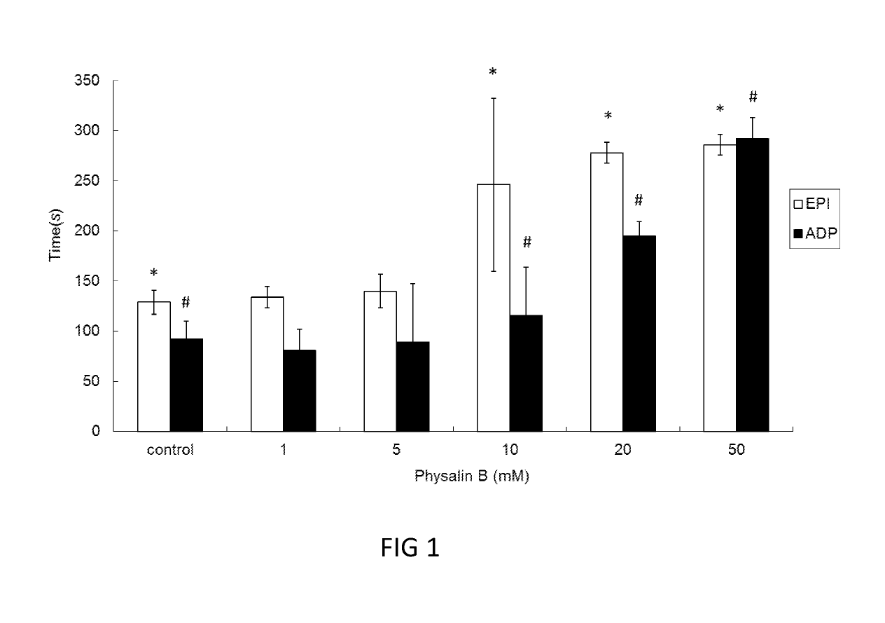

The PFA-100 system is a platelet function analyzer designed to measure platelet-related primary hemostasis. The system uses two disposable cartridges: a collagen/epinephrine (CEPI) and a collagen/ADP (CADP) cartridge. In both CEPI and CADP triggered plug formation conditions, samples pretreated with 10 μM physalin B exhibited statistically significant prolonged closure time, and the inhibitory effect was more profound when collagen-epinephrine was employed as the stimulus (FIG. 1), with the closure time (i.e., the time required for platelets to aggregate and close the aperture) twice longer than that of the control condition (125 sec for control, and 260 sec for physalin B treatment). The results demonstrated that physalin B may affect platelet aggregation and thus prevents plug formation.

1.2 the VerifyNow P2Y12 System

The VerifyNow P2Y12 system measures the rate and extent of changes in light transmittance caused by platelets aggregating in whole blood samples. Thus, samples with inhibited platelets (e.g., platelets treated with any anti-coagulant) produce low levels of light transmittance, while samples containing normally functioning platelets will deliver a higher level of transmittance. VerifyNow system is widely used for the assessment of the reactivity of platelets toward any anti-platelet agent, such as physalin B of the present invention, aspirin; clopidogrel, prasugrel and GP IIb/IIIa inhibitors. Results are summarized in Table 1.

In contrast to negative controls, the aspirin reaction unit (ARU) values obtained from blood samples treated with either 20 or 50 μM physalin B were both below 550 criteria (ARU=469 and 366, respectively), and were similar to that of therapeutic effective aspirin treatment (ARU=404, Table 1). As to the inhibition of P2Y12 receptor activation pathway, physalin B had mild effect at 20 μM, but significantly inhibited the activation of P2Y12 pathway at 50 μM, as the P2Y12 reaction unit (PRU) fell far below the normal range (PRU=65, Table 1). Ticagrelor and clopidogrel served as positive controls in this study. Ticagrelor reversibly inhibited the platelet P2Y12 receptors, which resulted in rapid inhibition of platelet activation and aggregation. Clopidogrel also acted on this receptor, however, since clopidogrel existed in the form of a “prodrug”, thus its effect on platelet inhibition tended to be slower and less consistent as compared with that of ticagrelor. Interestingly, the value of physalin B at 20 μM (PRU=226) was similar to clopidogrel treatment (PRU=263). Furthermore, since P2Y12 is the receptor responsible for ADP activator, thus physalin B's inhibitory effect on P2Y12 pathway is consistent with its interference on CADP induced platelet aggregation (FIG. 1). Although profound effects on arachidonic acid and P2Y12 pathways were detected, physalin B apparently did not interfere with GPIIb/IIIa receptor activity. Physalin B treated samples yielded similar platelet aggregation unit (PAU) values as that of the negative control one (PAU=144 and 161, respectively), which was in contrast to the potent, synthetic non-peptide GP IIb/IIIa receptor antagonist aggrastat (PAU=1, Table 1). Taken together, physalin B may modulate the two major upstream platelet activation pathways respectively triggered by arachidonic acid and ADP, but exerts little or no effect on the later platelet aggregation event, namely binding of platelet to fibrinogen through GPIIb/IIIa receptors.

TABLE 1
Antiplatelet function test using the VerifyNow system.

| Samples | Aspirin (ARU) | P2Y12 (PRU) | GPIIb/IIIa (PAU) |
|---|---|---|---|
| No Treatment | 660 | 314 | 161 |
| Treatment | | | |
| Aspirin | 404 | — | — |
| Ticagrelor | — | 1 | — |
| Clopidogrel | — | 263 | — |
| Aggrastat | — | — | 1 |
| 20 μM Physalin B | 469 | 226 | 144 |
| 50 μM Physalin B | 366 | 65 | 129 |

The degree of aggregation on an arachidonic acid pathway was reported in aspirin reaction units (ARU).
The P2Y12 receptor activation was represented as the P2Y12 reaction units (PRU).
The platelet glycoprotein (GP) IIb/IIIa receptors activity was reported as platelet aggregation units (PAU).

### Example 2 In Vivo Anti-Platelet Effects of Physalin B

The anti-platelet effects of Physalin B in live animals were investigated by measuring the tail bleeding time and the formation of thrombosis in microvessels in the experimental animals.

2.1 Effect of Physalin B on Tail Bleeding Time

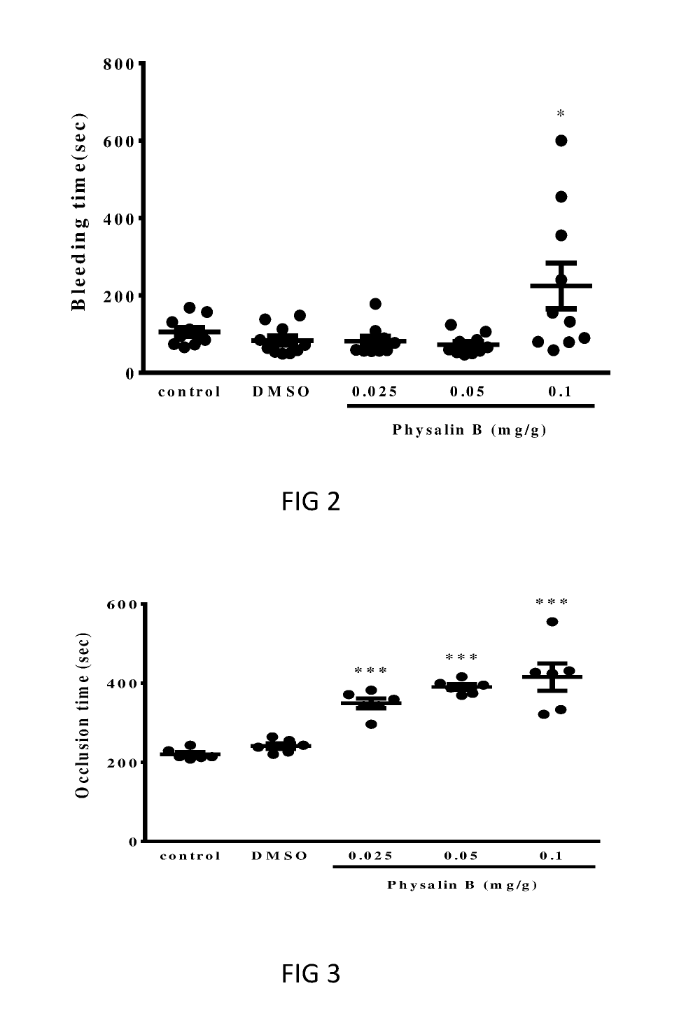

Briefly, the test mice were randomly divided into three groups, in which the control mice received intraperitoneal injection of normal saline, whereas mice in the vehicle and the test groups respectively received intraperitoneal injection of the vehicle (i.e., DMSO) and Physalin B (i.e., 0.025, 0.05 or 0.1 mg/g body weight). A cut (about 2-3 mm in length) was incised on the tail vein of each mice (about 3-5 cm from the tip of the tail) 30 minutes after the indicated treatment, then the blood was collected into a warm saline, in which the volume and the time to cessation of bleeding were both measured. Bleeding time was defined as the time in which the first cessation of bleeding was observed. Results are illustrated in FIG. 2.

The bleeding times for the control mice and the DMSO vehicle treated mice were about 105.6±11.3 sec and 83.1±11.7 sec, respectively. However, for mice that received 0.1 mg/Kg treatment of Physalin B, the bleeding time increased significantly to 224.4±58.9 sec. The results indicated that Physalin B is capable of suppressing the aggregation or activation of platelets.

2.2 Effect of Physalin B on the Formation of Thrombosis

In this example, the effects of Physalin B on the aggregation of platelets were investigated by the measurement of the occlusion time for irradiation induced platelet plug formation. Briefly, mice were respectively treated with normal saline, DMSO vehicle, and Physalin B (i.e., 0.025, 0.05, or 0.1 mg/g), then the mesentertic venules were selected for irradiation to induce microthrombus formation. The occlusion time was defined as the time in which the platelet plug was first observed. Results are illustrated in FIG. 3.

The occlusion times for the control mice and the DMSO vehicle treated mice were about 220.0±5.4 sec and 241±6.8 sec, respectively. However, when mice received 0.025, 0.05, and 0.1 mg/g treatment of Physalin B, the occlusion time increased significantly to 349±12.3, 390±7, and 415±34.5 secs, respectively. The results confirmed the observation in Examples 1 and 2.1 that Physalin B is capable of suppressing the aggregation or activation of platelets.

### Example 3 Cytotoxic Effect of Physalin B on Various Types of Cells

3.1 Cytotoxic Effect of Physalin B on Platelets

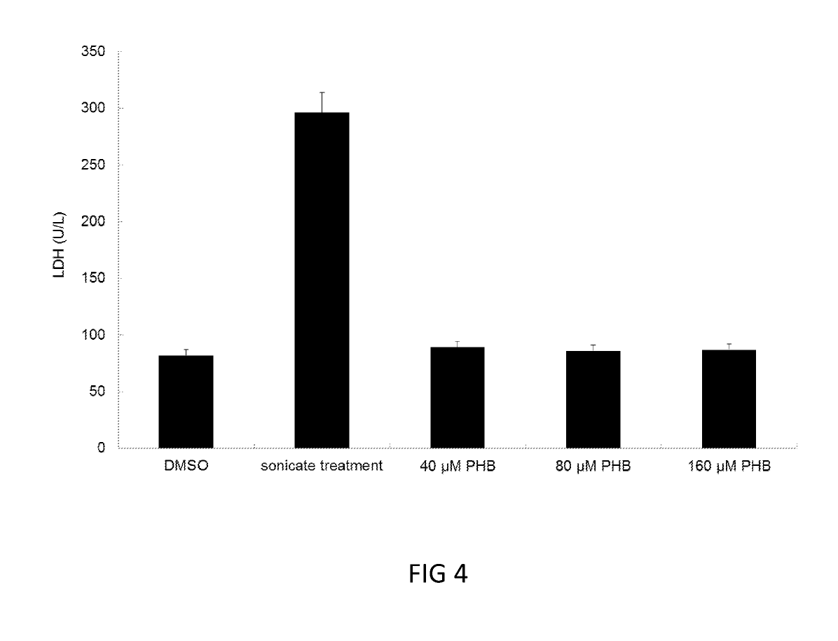

In this example, the cytotoxic effect of physalin B on platelets was investigated by measuring the activity of lactate dehydrogenase (LDH) released from the ruptured platelets. The platelets were exposed to various concentrations of physalin B (PHB) or DMSO, which was used as the vehicle control, while sonication ruptured platelets served as a positive control. The final concentration of DMSO in the test medium was less than 0.1%. Results are depicted in FIG. 4.

As the data indicated, no obvious LDH release was detected in platelets treated with up to 160 μM physalin B. Accordingly, it is reasonably to conclude that physalin B possess no cytotoxic effect toward platelets at the concentration below 160 μM.

3.2 Cytotoxic Activity of Physalin B on HUVEC and EPCs

The cytotoxicity of physalin B on EPCs and HUVECs were examined in this example.

Two types of EPCs (i.e., early EPCs and late EPCs) were obtained in accordance with procedures described in the “Materials and Methods” section. Briefly, mononuclear cells (MNCs) were first isolated from peripheral blood and subsequently plated on 6-well tissue culture plates pre-coated with human fibronectin. Small colonies started to appear after 1-2 weeks in culture. The initially seeded cells were respectively in round shapes. After 5 to 10 days, attached cells appeared in clusters. After 2 to 4 weeks, cultured cells appeared to have a smooth cytoplasmic outline, and were firmly attached onto the plate in addition, they exhibited a cobblestone appearance similar to that of HUVECs when they divided. These cells replicated rapidly and formed a colony, and were termed late EPCs, which were a monolayer with almost full confluence (data not shown).

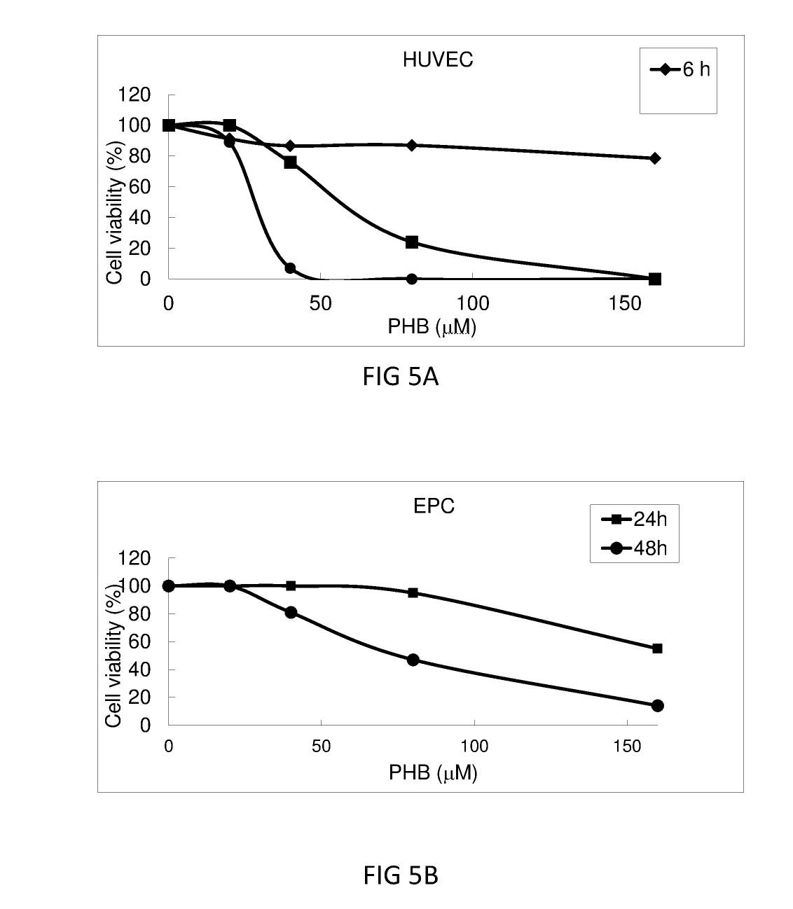

The in vitro cytotoxic effect of physalin B on HUVEC and EPCs were respectively determined by MTT assay, in which cells were exposed to various concentrations of physalin B (i.e., 0, 20, 40, 80 and 160 μM). Results are illustrated in FIGS. 5A and 5B.

FIG. 5A depicts the time-dependent and dose-dependent growth inhibition of physalin B on HUVECs; while the same for EPCs were depicted in FIG. 5B. Although HUVEC and late EPCs were both susceptible to physalin B at relative high concentration (40 μM) and long incubation time (48 hr), late EPCs were less vulnerable to the cytotoxic effect of physalin B, as compared with that of HUVECs. EPCs were less susceptible to physalin B than that of HUVECs, in which the IC₅₀ for EPCs and HUVECs treated with physalin B for 48 hrs were 76 and 30 μM, respectively.

Taken together, the results clearly indicated that physalin B has mild cytotoxic effect towards HUVEC and/or EPCs, and no apparent adverse effects towards EPCs and HUVECs and therefore their physiological functions.

### Example 4 Physalin B Reduces the Adhesion of THP-1 Cells to HUVEC Monolayers

In this example, the effects of physalin B on the adhesion of monocytic leukocyte (THP-1) on HUVECs under inflammatory condition were investigated.

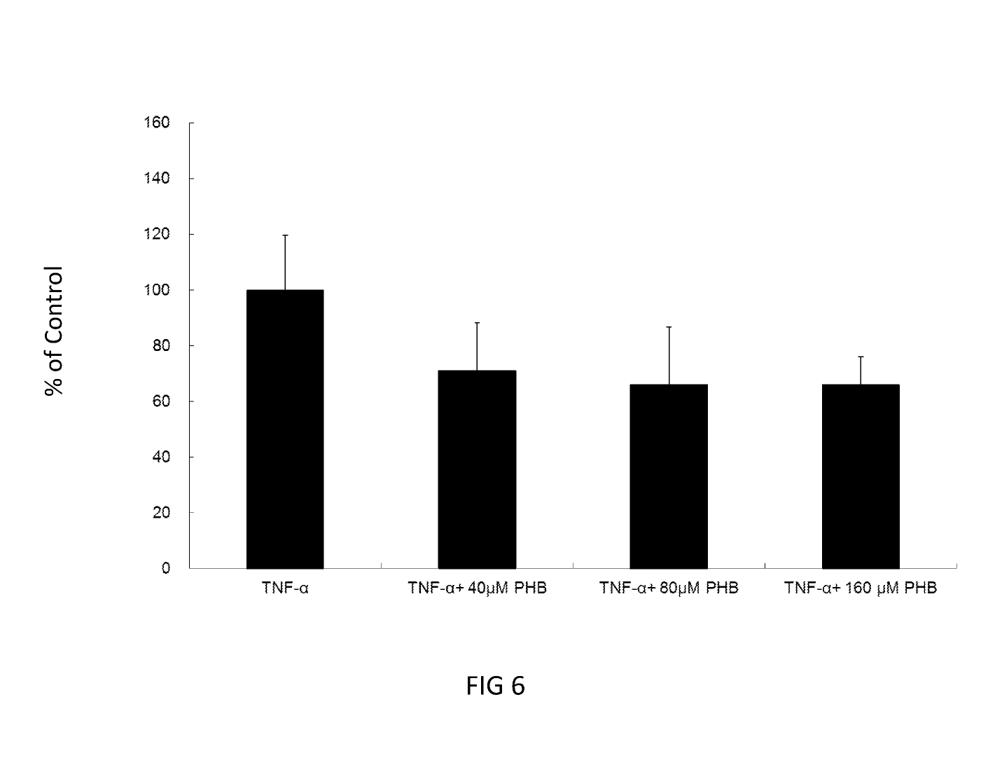

The in vitro adhesion of THP-1 to HUVECs was monitored by use of a fluorescence dye, Calcein-AM, which stains only the viable cells. Briefly, THP-1 cells were labeled with calcein-AM first, then co-cultured with physalin B pre-treated HUVECs in the presence of TNF-α, which was added to induce inflammation. The fluorescence intensity at 525 nm was then measured. Quantified results are presented in FIG. 6.

In general, the intensity of fluorescence remaining in the THP-1 cells was proportional to the number of THP-1 attached to HUVECs. The fluorescent cells could be either visualized under fluorescent microscope or quantified using a fluorescence reader. As depicted in FIG. 6, treating the TNF-α-exposed cells with 80 μM physalin B resulted in about 35% inhibition on the adhesion of THP-1 to HUVECs, as compared to that treated with TNF-α alone (P<0.05). This finding indicated that physalin B may also possess an anti-inflammatory effect through the inhibition of monocytic leukocytes adhesion to epithelial cells.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1-8. (canceled)

9. A method for treating a subject having or suspected of having a disease resulting from platelet aggregation comprising administering to the subject an effective amount of physalin for alleviating or ameliorating the symptoms associated with the disease.

10. The method of claim 9, wherein the physalin is physalin B.

11. The method of claim 9, wherein the physalin is administered to the subject in the amount of 0.001-100 mg/Kg.

12. The method of claim 11, wherein the physalin is administered to the subject in the amount of 0.001-10 mg/Kg.

13. The method of claim 12, wherein the physalin is administered to the subject in the amount of 0.01-10 mg/Kg.

14. The method of claim 9, further comprising administering an anti-coagulant to the subject.

15. The method of claim 14, wherein the anti-coagulant is selected from the group consisting of, abciximab, apixaban, aspirin, clopidogrel, dipyridamole, edoxaban, eptifibatide, rivaroxaban, tirofiban, ticlopidine, warfarin, and vitamin K.

16. The method of claim 9, wherein the disease is a thrombotic disorder.

17. The method of claim 16, wherein the thrombotic disorder is selected from the group consisting of, abrupt vessel closure following angioplasty or stent placement, atherothrombosis, acute thrombotic stroke, myocardial infarction, thrombosis resulted from periphery vascular surgery, unstable angina, and venous thrombosis.

18. The method of claim 9, wherein the subject is human.

19. The method of claim 9, wherein the physalin is applied as a coating on an implantable device.

20-32. (canceled)
